(19) United States
(12) Patent Application Publication
(10) Pub. No.: US 2012/0269870 A1
(43) Pub. Date: Oct. 25, 2012

(54) SWITCHABLE POLYMERS AND SURFACES WITH REVERSIBLY SWITCHABLE PROPERTIES

(75) Inventors: Shaoyi Jiang, Redmond, WA (US); Zhiqiang Cao, Somerville, MA (US)

(73) Assignee: University of Washington through its Center for Commercialization, Seattle, WA (US)

(21) Appl. No.: 13/453,809

(22) Filed: Apr. 23, 2012

Related U.S. Application Data
(60) Provisional application No. 61/477,807, filed on Apr. 21, 2011.

(51) Int. Cl. C08L 39/04; A61K 9/00; C08F 8/00; C08F 122/22; C08F 126/06

(52) U.S. Cl. 424/400; 526/312; 526/260; 525/204; 525/386

(57) ABSTRACT

Reversibly switchable polymers, surfaces that include the polymers, and methods for making and using the polymers and surfaces. The switchable polymers are non-fouling in their zwitterionic form and are antimicrobial in their cationic form.

### CROSS-REFERENCE TO RELATED APPLICATION

This application claims the benefit of U.S. Provisional Application No. 61/477,807, filed Apr. 21, 2011, expressly incorporated herein by reference in its entirety.

### STATEMENT OF GOVERNMENT LICENSE RIGHTS

This invention was made with U.S. government support under HDTRA1-10-1-0074 awarded by the Defense Threat Reduction Agency (DTRA), and under N000140910137 awarded by the Office of Naval Research (ONR). The U.S. Government has certain rights in the invention.

### BACKGROUND OF THE INVENTION

Bacteria are common biological threats. Antimicrobial and non-fouling coatings are conventional approaches to detoxify these microorganisms and prevent their contamination. In the antimicrobial approach, cationic antimicrobial surfaces can be used to kill bacteria. However, bio-macromolecules and dead microorganisms can easily attach to the surfaces due to their opposite charges and eventually block their antimicrobial functions. To suppress the accumulation of microorganisms onto a surface, another conventional method is to graft surfaces with non-fouling polymers such as zwitterionic polymers. Although zwitterionic coatings can reduce initial attachment and delay colonization of microbes on surfaces, non-fouling polymer coatings alone cannot kill bacteria and result in the failure of the surface once pathogenic microbes are attached onto a surface. Thus, materials bearing both antimicrobial and non-fouling capabilities are highly desirable to resist bacterial adhesion/biofilm formation for a prolonged period of time.

A switchable polymer surface coating has been described. In this coating, a cationic precursor of a zwitterionic compound effective for killing bacteria is irreversibly switched to a non-fouling zwitterionic surface by hydrolysis, thereby releasing all attached microbes. However, this surface is a “one time” switch; once the surface has been hydrolyzed to its non-fouling form, the surface cannot be regenerated to its antimicrobial form.

Despite the advances in the development of non-fouling and antimicrobial materials noted above, a need exists for materials bearing both antimicrobial and non-fouling capabilities to resist bacterial adhesion/biofilm formation for a prolonged period of time with capabilities for full surface regeneration. The present invention seeks to fulfill this need and provides further related advantages.

### SUMMARY OF THE INVENTION

In one aspect, the present invention provides reversibly switchable polymers and compounds.

In one embodiment, the invention provides a polymer having formula (I):

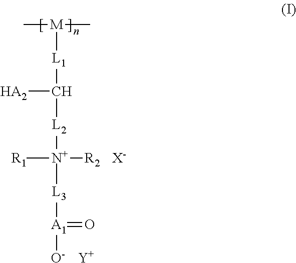

wherein

M is a monomeric repeating unit;

L₁ is a first linker that covalently couples the methine carbon to the repeating unit;

A₂ is O, NR₃, or S, wherein R₃ is selected from the group consisting of hydrogen, C1-C6 alkyl, and C6-C12 aryl;

R₁ and R₂ are independently selected from the group consisting of hydrogen, C1-C6 alkyl, and C6-C12 aryl, or taken together with the nitrogen to which they are attached form a cationic center;

L₂ is a second linker that covalently couples the methine carbon to the cationic center;

A₁(=O)—O⁻ is an anionic center, wherein A₁ is selected from the group consisting of C, Si, SO, and POH;

L₃ is a third linker that covalently couples the cationic center to the anionic center;

X⁻ is a counter ion associated with the cationic center;

Y⁺ is a counter ion associated with the anionic center; and

n is an integer from 1 to about 10,000.

Suitable monomeric repeating units include repeating units for polyesters, polyamides, poly(amino acids), polyimides, polycarbonates, polysiloxanes, polyurethanes, polyphosphazenes, acrylic polymers, amino resins, epoxy resins, phenolic resins, and alkyd resins. A representative monomeric repeating unit is —C(R₄)(R₅)—C*(R₆)—, wherein R₄, R₅, and R₆ are independently selected from the group consisting of hydrogen, fluorine, substituted and unsubstituted C1-C6 alkyl, and substituted and unsubstituted C6-C12 aryl, and wherein * represents the point of attachment to L₁.

In one embodiment, the cationic center is formed by taking R₁ and R₂ together with the nitrogen to which they are attached to provide a quaternary ammonium, a imidazolium, a triazolium, a pyridinium, or amorpholinium center.

In another embodiment, the invention provides a polymer having formula (II):

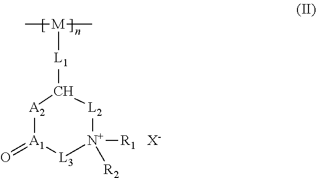

wherein

M is a monomeric repeating unit;

L₁ is a first linker that covalently couples the methine carbon to the repeating unit;

A₂ is O, NR₃, or S, wherein R₃ is selected from the group consisting of hydrogen, C1-C6 alkyl, and C6-C12 aryl;

R₁ and R₂ are independently selected from the group consisting of hydrogen, C1-C6 alkyl, and C6-C12 aryl, or taken together with the nitrogen to which they are attached form a cationic center;

L₂ is a second linker that covalently couples the methine carbon to the cationic center;

L₃ is a third linker that covalently couples the cationic center to A₁;

A₁ is selected from the group consisting of C, Si, SO, and POH;

X⁻ is a counter ion associated with the cationic center;

Y⁺ is a counter ion associated with the anionic center; and

n is an integer from 1 to about 10,000.

In another aspect, the present invention provides reversibly switchable compounds.

In one embodiment, the invention provides a compound having formula (III):

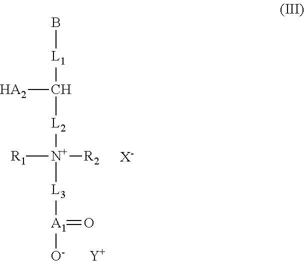

wherein

B is a polymerizable group or an adhesive group;

L₁ is a first linker that covalently couples the methine carbon to the repeating unit;

A₂ is O, NR₃, or S, wherein R₃ is selected from the group consisting of hydrogen, C1-C6 alkyl, and C6-C12 aryl;

R₁ and R₂ are independently selected from the group consisting of hydrogen, C1-C6 alkyl, and C6-C12 aryl, or taken together with the nitrogen to which they are attached form a cationic center;

L₂ is a second linker that covalently couples the methine carbon to the cationic center;

A₁(=O)—O⁻ is an anionic center, wherein A₁ is selected from the group consisting of C, Si, SO, and POH;

L₃ is a third linker that covalently couples the cationic center to the anionic center;

X⁻ is a counter ion associated with the cationic center; and

Y⁺ is a counter ion associated with the anionic center.

In one embodiment, the invention provides a compound having formula (IV):

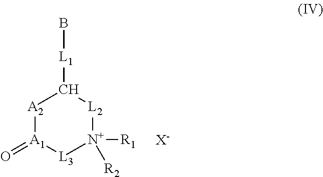

wherein

B is a polymerizable group or an adhesive group;

L₁ is a first linker that covalently couples the methine carbon to the repeating unit;

A₂ is O, NR₃, or S, wherein R₃ is selected from the group consisting of hydrogen, C1-C6 alkyl, and C6-C12 aryl;

R₁ and R₂ are independently selected from the group consisting of hydrogen, C1-C6 alkyl, and C6-C12 aryl, or taken together with the nitrogen to which they are attached form a cationic center;

L₂ is a second linker that covalently couples the methine carbon to the cationic center;

L₃ is a third linker that covalently couples the cationic center to A₁;

A₁ is selected from the group consisting of C, Si, SO, and POH;

X⁻ is a counter ion associated with the cationic center; and

Y⁺ is a counter ion associated with the anionic center.

In another aspect, the invention provides surfaces that include the switchable polymer and compounds. In one embodiment, the invention provides a surface of a substrate, wherein the surface comprises a layer of the polymers of formula (I). In another embodiment, the invention provides a surface of a substrate, wherein the surface comprises a layer of the polymers of formula (II). In a further embodiment, the invention provides a surface of a substrate, wherein the surface comprises a layer of the polymers of formulas (I) and (II). In one embodiment, the invention provides a surface of a substrate, wherein the surface comprises a layer of the compounds of formula (III). In another embodiment, the invention provides a surface of a substrate, wherein the surface comprises a layer of the compounds of formula (IV). In a further embodiment, the invention provides a surface of a substrate, wherein the surface comprises a layer of the compounds of formulas (III) and (IV).

In certain embodiments, the surface of the invention further include a functional molecule covalently coupled to one or more of the polymers, or one or more of the compounds, of the layers.

In further aspects, the invention provides methods for converting a surface of a substrate from a zwitterionic polymer surface to a cationic polymer surface, and for converting a surface of a substrate from a cationic polymer surface to a zwitterionic polymer surface. In one embodiment, the invention provides a method for converting a surface of a substrate from a zwitterionic polymer surface to a cationic polymer surface, comprising contacting a surface of a substrate having a zwitterionic polymer surface with acidic conditions to provide a cationic polymer surface, wherein the zwitterionic polymer surface comprises a layer of zwitterionic polymers of formula (I), and wherein the cationic polymer surface comprises a layer of cationic polymers of formula (II). In another embodiment, the invention provides a method for converting a surface of a substrate from a cationic polymer surface to a zwitterionic polymer surface, comprising contacting a surface of a substrate having a cationic surface with aqueous or basic conditions to provide a zwitterionic polymer surface, wherein the cationic polymer surface comprises a layer of cationic polymers of formula (II), and wherein the zwitterionic polymer surface comprises a layer of zwitterionic polymers of formula (I). In certain embodiments, the surfaces further include a functional molecule covalently coupled to one or more of the polymers of the layers.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 illustrates representative reversible switchable polymers and surfaces of the invention: switchable CB-Ring/CB—OH surface. CB-Ring is able to kill bacteria under dry conditions due to its cationic lactone ring. Under wet conditions, CB-Ring is hydrolyzed to zwitterionic CB—OH, which is ultra-low fouling and able to release adsorbed dead bacteria. CB-Ring can be regenerated from CB—OH in acidic, non-aqueous environment.

FIG. 2 illustrates the chemical structures of representative CB-Ring and CB—OH monomers.

FIG. 3 compares the bactericidal activity of various surfaces against E. coli K12. The CB—OH surface was prepared via ATRP with ATRP initiators on a gold surface. The CB-Ring surface is prepared by dipping CB—OH surface in trifluoroacetic acid for 1 h. All surfaces were washed with acetonitrile to remove residual acids and dried before contacting bacteria. Bacteria were allowed to stand on the surface for 1 h in the air, and then cultured for colonies growing overnight.

FIG. 4 illustrates the properties of representative switchable polymer surfaces of the invention: CB—OH (monomer shown) illustrates the “non-sticky” state that resists biomolecular adsorption and CB-Ring (monomer shown) represents the “sticky” state that covalently couples to amine-containing biomolecules. Unreacted CB-Ring groups can then be converted back into an ultra-low fouling surface.

FIG. 5 compares the equilibrium kinetics between CB—OH and CB-Ring structures in three deuterated solvent environments: (1) TFA (TFA-d) □, (2) pH 7.3 buffer ∘ (200 mM Na₂CO₃ in D₂O titrated with DCl), and (3) pH 10 buffer Δ (200 mM Na₂CO₃ in D₂O titrated with DCl). Conversion was calculated via ¹H NMR using a ratio of the characteristic peak for CB-Ring (1H, CH₂═C(CH₃)COOCH₂CH(O—)CH₂—) and a common peak of both structures (6H, —CH₂N(CH₃)₂CH₂—).

FIG. 6 is an image of a 6×12 protein array that includes a thin polymer coating of a representative switchable polymer of the invention. Anti-hCG and anti-Salm were printed onto the array as indicated. The dark background was ultra-low fouling.

FIG. 7 illustrates the specific detection of hCG using the array illustrated in FIG. 6. Response was observed for only the anti-hCG printed sites; no response was observed for the background or the control antibody (anti-Salm).

FIG. 8 illustrates the chemical structures of cationic CB-Ring monomer, zwitterionic CB—OH monomer, and cationic C8N monomer.

FIG. 9 compares the bactericidal activity of the surfaces prepared by polymerization of the monomers illustrated in FIG. 8 (CB-Ring, TFA; CB-Ring, HAc; CB—OH; and CBN) against E. coli K12 under dry conditions. The Y axis represents the percentage of live E. coli K12 colonies grown relative to the number of colonies grown on CB—OH control (n=3). Note that zwitterionic CB—OH does not kill the bacteria.

FIG. 10 compares bacterial cell density of E. coli K12 on the surfaces (CB-Ring, TFA; CB-Ring, HAc; CB—OH; and CBN) before and after the releasing procedure (gently shaking in PBS for 1 h) (n=3). Initially (before release), bacteria were equally sprayed on CB-Ring, CB—OH and C8N surfaces and dried for 15 min. After the releasing procedure, the remaining bacteria coverage was recorded. The bacteria coverage for CB-Ring and CB—OH surfaces was the same as C8N, but could not be directly measured because staining required wet conditions that released bacteria on CB-Ring and CB—OH surfaces.

FIG. 11 compares bacterial cell density of E. coli K12 on the surfaces (CB-Ring, CB—OH, and CBN) after incubating with the bacteria under wet conditions (10⁸ cells mL⁻¹ for 0.5 h) (n=3). CB-Ring was converted to CB—OH during the incubation.

FIG. 12 is a schematic illustration of the preparation of a representative monomer, CB—OH, useful for making a switchable polymer of the invention.

FIG. 13 is a SPR sensor-gram showing the ultra-low fouling properties of the CB—OH polymer brushes formed via SI-ATRP on gold substrates. Surface coverage for Lyz (lysozyme) and Fib (fibrinogen) was below the detection limit (about 0.3 ng/cm²) while undiluted human plasma resulted in 3.1±1.0 ng/cm².

FIG. 14 compares immobilization levels for anti-hCG at pH 6 and 10 on a protein array that includes a thin polymer coating of a representative switchable polymer of the invention with anti-hCG printed onto the array. The image is a typical SPR image where the brighter sites indicate a higher level of antibody immobilization.

FIG. 15 illustrates the conversion kinetics from CB—OH monomer to CB-Ring monomer in [D] acetic acid. At various time points, the molar composition of CB-Ring was calculated by ¹H NMR spectroscopy using the ratio of the characteristic peak for CB-Ring (m, 1H, CH₂═C(CH₃)COOCH₂CH(O—)CH₂—) at δ=5.4 ppm and a common characteristic peak (6H, —CH₂N(CH₃)₂CH₂—) for both CB-Ring and CB—OH at δ=3.55 and 3.45 ppm, respectively.

FIG. 16 illustrates the conversion kinetics from CB-Ring monomer to CB—OH monomer in pH 7.3 buffer made from 200 mM Na₂CO₃ in D₂O titrated with DCl. At various time points, the molar composition of CB-Ring was calculated by ¹H NMR spectroscopy using the ratio of the characteristic peak for CB-Ring (m, 1H, CH₂═C(CH₃)COOCH₂CH(O—)CH₂—) at δ=5.4 ppm and a common characteristic peak (6H, —CH₂N(CH₃)₂CH₂—) for both CB-Ring and CB—OH at δ=3.28 ppm. The final conversion to CB—OH within 160 min was about 93%. Because the integration error for ¹H NMR can be as large as 10%, a value of 93% represents essentially complete conversion.

FIG. 17 illustrates a proposed mechanism for the equilibrium between CB—OH and CB-Ring under acid conditions. The conversion from CB—OH to CB-ring is catalyzed by the protonation of the carboxylate group leading to an intramolecular Fischer esterification or lactonization involving the neighboring hydroxyl group. A strong acid is the ideal catalyst for the reaction as is the case for TFA which gave an almost complete conversion to CB-ring in 1 h; on the other hand a weak acid such as HAc yielded only a 50% conversion after 20 h. Such a difference is due to the stronger ability of TFA to both protonate CB—OH and consume the H₂O generated (by forming the oxonium ion H₃O⁺), thus significantly displacing the reaction equilibrium towards the CB-ring via Le Chatelier's principle. HAc is 105-fold less acidic than TFA and has a pKa much closer to that of CB—OH, which leads to a much smaller fraction of CB—OH being protonated at any given time compared to the case in TFA, resulting in a much slower lactonization rate.

FIG. 18 illustrates a proposed mechanism for the equilibrium between CB-Ring and CB—OH under neutral and basic conditions.

FIG. 19 compares film thickness of CB—OH after each cycle of the treatment (mean±SD; n=3). For one cycle of treatment, the CB—OH surface was dipped into acetic acid for 20 h for CB-Ring generation followed by 2 h incubation with PBS for the hydrolysis back to CB—OH.

FIG. 20 compares fluorescence microscopy images of E. coli K12 (light spots) attached to CB-Ring (TFA), CB-Ring (HAc), CB—OH, and CBN surfaces before and after the releasing procedure. See FIG. 10.

FIG. 21 compares fluorescence microscopy images of attached E. coli K12 (light spots) after incubating CB-Ring, CB—OH, and CBN surfaces with bacteria in wet conditions. See FIG. 11.

### DETAILED DESCRIPTION OF THE INVENTION

The present invention provides reversibly switchable polymers and compounds and surface coatings that include reversibly switchable polymers and compounds. The switchable polymers and compounds are non-fouling in their zwitterionic form and are anti-microbial in their cationic form. The polymers and compounds are reversibly switchable between their zwitterionic and cationic forms by simple pH adjustment.

In another aspect, the invention provides surfaces that include the switchable polymer and compounds. By virtue of the switchable polymers and compounds, the surfaces of the invention are reversibly switchable between their zwitterionic (non-fouling) and cationic (anti-microbial) forms by simple pH adjustment. The switchable polymers and compounds can covalently coupled to the surfaces or associated to the surfaces through non-covalent attachment (e.g., ionic or hydrophobic association). The switchable polymers can be grafted from the surface by polymerization of suitable monomers from surface attached initiators.

Methods for making and using the switchable polymers and compounds and switchable surfaces including the polymers and compounds are also provided.

Switchable Polymers and Switchable Compounds

In one aspect, the present invention provides switchable polymers and switchable compounds.

In one embodiment, the invention provides switchable zwitterionic polymers having formula (I).

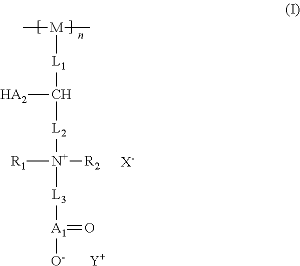

In another embodiment, the invention provides switchable cationic polymers having formula (II):

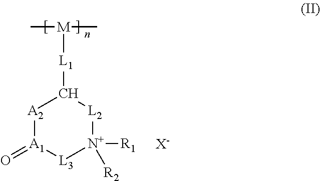

As noted above, the zwitterionic polymers and the cationic polymers are reversibly switchable as illustrated below.

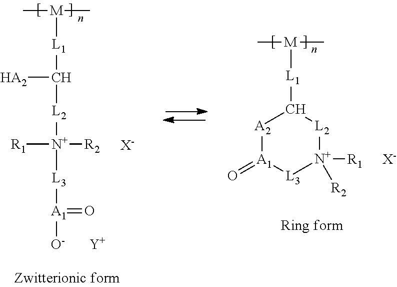

The polymers of the invention include the monomeric repeating units illustrated above. It will be appreciated that the terminal groups of the polymers, not illustrated, will depend on the nature of the polymerization reaction utilized to prepare the polymers. These polymerization reactions and their terminal groups are known in the art.

In a further embodiment, the invention provides switchable zwitterionic compounds having formula (III).

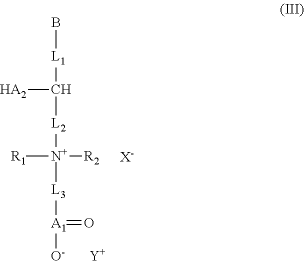

In another embodiment, the invention provides switchable cationic compounds having formula (IV).

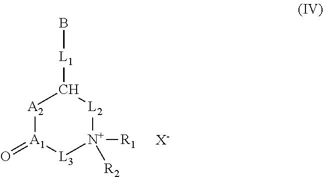

It will be appreciated that the reversibility of the zwitterionic form (open) and cationic form (ring) illustrated above for the switchable polymers of the invention is equally applicable to the switchable compounds of the invention.

The following is a description of the components of the switchable polymers and switchable compounds of the invention illustrated above.

For the polymers, M is a monomeric repeating unit. The monomeric repeating unit is derived from the monomer that is polymerized to provide the polymer. Suitable monomeric repeating units include repeating units for polyesters, polyamides, poly(amino acids), polyimides, polycarbonates, polysiloxanes, polyurethanes, polyphosphazenes, acrylic polymers, amino resins, epoxy resins, phenolic resins, and alkyd resins. In one embodiment, the monomeric repeating unit is —C(R₄)(R₅)—C*(R₆)—, wherein R₄, R₅, and R₆ are independently selected from the group consisting of hydrogen, fluorine, substituted and unsubstituted C1-C6 alkyl, and substituted and unsubstituted C6-C12 aryl, and wherein * represents the point of attachment to L₁. In certain embodiments, M is —CH₂—C*(CH₃)—.

For the switchable compounds, B is either a polymerizable group or an adhesive group.

When B is a polymerizable group, the compounds of formulas (III) and (IV) are monomers. Suitable polymerizable groups include those known in the art. Representative polymerizable groups are effective for preparing polyesters, polyamides, poly(amino acids), polyimides, polycarbonates, polysiloxanes, polyurethanes, polyphosphazenes, acrylic polymers, amino resins, epoxy resins, phenolic resins, and alkyd resins. In one embodiment, the polymerizable group is C(R₄)(R₅)═C(R₆)(X), wherein R₄, R₅, and R₆ are independently selected from the group consisting of hydrogen, fluorine, substituted and unsubstituted C1-C6 alkyl, and substituted and unsubstituted C6-C12 aryl, and wherein X represents the polymer's pendant groups illustrated in formulas (I)-(IV). In certain embodiments, B is CH₂═C(CH₃)(X).

The adhesive group renders the compounds useful for direct attachment to surfaces thereby imparting advantageous properties to those surfaces. In this aspect, the compound is grafted to the surface through the interaction of the compound's adhesive group and the surface. Suitable adhesive groups include dihydroxyphenyl groups. The dihydroxyphenyl group is a biomimetic adhesive group that allows the compound to adhere to a variety of surfaces thereby immobilizing the compound on the surface. In one embodiment, the dihydroxyphenyl group is a 3,4-dihydroxyphenyl group (i.e., a catechol group). In certain embodiments, the compound includes a 3,4-dihydroxyphenyl group derived from 3,4-dihydroxyphenyl alanine (i.e., DOPA). Adhesive groups can be incorporated into the polymers of the invention and utilize to non-covalently attach switchable polymers to a variety of surfaces. The preparation of polymers having adhesive groups and their use for immobilizing the polymers is described in U.S. Patent Application Publication No. 2011/0105712, expressly incorporated herein by reference in its entirety.

The methine carbon is the carbon atom bearing the single hydrogen atom (CH) and that is bonded to L₁, L₂, and A₂.

A₂ is O, NR₃, or S, wherein R₃ is selected from the group consisting of hydrogen, C1-C6 alkyl, and C6-C12 aryl. In one embodiment, A₂ is O.

L₁ is a first linker that covalently couples the methine carbon to the monomeric repeating unit. Representative L₁ groups include —C(═O)O—(CH₂)ₙ— and —C(═O)NH—(CH₂)ₙ—, where n is an integer from 1 to 20 (e.g., 3).

L₂ is a second linker that covalently couples the methine carbon to the cationic center. L₂ can be a C1-C3 alkylene chain. Representative L₂ groups include —(CH₂)ₙ—, where n is 0, 1, 2, or 3.

L₃ is a third linker that covalently couples the cationic center to A₁. L₃ can be a C1-C3 alkylene chain. Representative L₃ groups include —(CH₂)ₘ—, where m is 1, 2, 3, or 4.

For L₂ and L₃, n+m≦4. In one embodiment, n=1 and m=1.

N⁺ is the cationic center. In certain embodiments, the cationic center is a quaternary ammonium (e.g., N bonded to L₂, R₁, R₂, and L₃). In addition to ammonium, other useful cationic centers (R₁ and R₂ taken together with N) include imidazolium, triazaolium, pyridinium, morpholinium, oxazolidinium, pyrazinium, pyridazinium, pyrimidinium, piperazinium, and pyrrolidinium.

R₁, R₂, and R₃ are independently selected from hydrogen, substituted and unsubstituted alkyl, and substituted and unsubstituted aryl groups. Representative alkyl groups include C1-C6 straight chain and branched alkyl groups. In certain embodiments, the alkyl group is further substituted with one of more substituents including, for example, an aryl group (e.g., —CH₂C₆H₅, benzyl). Representative aryl groups include C6-C12 aryl groups including, for example, phenyl. For certain embodiments of the above formulas, R₁ and R₂ are taken together with the nitrogen to which they are attached to form the cationic center. In one embodiment, R₁ and R₂ are C1-C6 alkyl. In another embodiment, R₁ and R₂ are C1-C3 alkyl. In a further embodiment, R₁ and R₂ are methyl.

A₁ is C, Si, SO, or POH. For the zwitterionic forms, A₁(=O)—O⁻ is the anionic center. In one embodiment, A₁ is C.

X⁻ is the counter ion associated with the cationic center. The counter ion can be the counter ion that results from the synthesis of the cationic polymers or the monomers (e.g., halides such as Cl⁻, Br⁻, and I⁻). The counter ion that is initially produced from the synthesis of the cationic center can also be exchanged with other suitable counter ions to provide polymers having controllable hydrolysis properties and other biological properties. Representative hydrophobic counter ions include carboxylates, such as benzoic acid and fatty acid anions (e.g., CH₃(CH₂)ₙCO2 where n=1-19); alkyl sulfonates (e.g., CH₃(CH₂)ₙSO₃⁻ where n=1-19); salicylate; lactate; bis(trifluoromethylsulfonyl)amide anion (N⁻(SO₂CF₃)₂); and derivatives thereof. Other counter ions also can be chosen from halides such as chloride, bromide, iodide, sulfate; nitrate; perchlorate (ClO₄); tetrafluoroborate (BF₄); hexafluorophosphate (PF₆); trifluoromethylsulfonate (SO₃CF₃); and derivatives thereof. Other suitable counter ions include hydrophobic counter ions and counter ions having therapeutic activity (e.g., an antimicrobial agent, such as salicylic acid (2-hydroxybenzoic acid), benzoate, and lactate.

Y⁺ is a counter ion associated with the anionic center. At each occurrence Y⁺ is a metal ion (e.g., sodium, lithium, potassium, calcium), ammonium ion (e.g., NR₄⁺, where each R is the same or different and selected from hydrogen, alkyl, and aryl), and organic ions.

Referring to formulas (I) and (II), n is an integer from 1 to about 10,000. When n is 1, formulas (I) and (II) are compounds having a single monomeric unit. When n is 2 to 9, formulas (I) and (II) are oligomers having from 2 to 9 monomeric repeating units. When n is 10 or greater, formulas (I) and (II) are polymers having 10 or greater monomeric repeating units. In certain embodiments, n is 1. In other embodiments, n is an integer from 2 to 9. In certain embodiments, n is an integer from about 10 to about 5,000. In other embodiments, n is an integer from about 10 to about 1,000.

For the polymers of the invention, the degree of polymerization (DP or n), number average molecular weight (Mₙ), and the ratio of weight average and number average molecular weights (M_{w}/Mₙ.), also known as polydispersity index, can vary. In one embodiment, the polymers of the invention have a degree of polymerization (n) from 10 to about 10,000. In one embodiment, n is from about 10 to about 1,000. In another embodiment, n is from about 100 to about 350. In one embodiment, the polymers of the invention have a number average molecular weight (Mₙ) of from about 200 to about 200,000. In one embodiment, Mₙ is from about 2,000 to about 100,000. In another embodiment, Mₙ is from about 20,000 to about 80,000. In one embodiment, the polymers of the invention have a ratio of weight average and number average molecular weight (M_{w}/Mₙ.) of from about 1.0 to about 2.0. In one embodiment, M_{w}/Mₙ. is from about 1.1 to about 1.5. In another embodiment, M_{w}/Mₙ. is from about 1.2 to about 2.0.

In certain embodiments, L₁ is —C(═O)O—CH₂—, L₂ is —CH₂—, L₃ is —CH₂—, A₁ is C, A₂ is O, and R₁ and R₂ are methyl.

In the above formulas, representative alkyl groups include C1-C30 straight chain and branched alkyl groups. In certain embodiments, the alkyl group is further substituted with one of more substituents including, for example, an aryl group (e.g., —CH₂C₆H₅, benzyl).

Representative aryl groups include C6-C12 aryl groups including, for example, phenyl including substituted phenyl groups (e.g., benzoic acid).

Switchable Surfaces

In another aspect of the invention, switchable surfaces are provided. Suitable switchable surfaces include one or more surfaces of a substrate.

In one embodiment, the invention provides a surface of a substrate, wherein the surface comprises a layer of polymers of formula (I).

In another embodiment, the invention provides a surface of a substrate, wherein the surface comprises a layer of polymers of formula (II).

In a further embodiment, the invention provides a surface of a substrate, wherein the surface comprises a layer of polymers of formulas (I) and (II).

In one embodiment, the invention provides a surface of a substrate, wherein the surface comprises a layer of compounds of formula (III).

In another embodiment, the invention provides a surface of a substrate, wherein the surface comprises a layer of compounds of formula (IV).

In a further embodiment, the invention provides a surface of a substrate, wherein the surface comprises a layer of compounds of formulas (III) and (IV).

Any of the surfaces described herein can further include a functional molecule covalently coupled to one or more of the polymers or compounds of the surface's layer. Functional molecules are molecules that impart a function to the surface. Suitable functional molecules include reporting molecules that have the capability of reporting the nature of the environment of the surface. Other suitable functional molecules include capture molecules that have the capability of binding molecules to the surface. Representative capture molecules include biomolecules. When the functional molecule is a biomolecule, such surfaces become biomolecularly functional, in addition to non-fouling and/or anti-microbial. Such biomolecule-containing surfaces are useful in protein arrays for a variety of screening purposes as well as sensing applications. Suitable biomolecules include proteins, peptides, polynucleic acids (e.g., DNAs and RNAs), polysaccharides, and small molecules. Representative biomolecules include enzymes and their substrates, receptors and their ligands, and antibodies and antibody fragments and their antigens.

The switchable polymers and switchable compounds of the invention can be advantageously used as coatings for the surfaces of a variety of devices including, for example, medical devices.

The switchable polymers and switchable compounds of the invention are advantageously used to coat surfaces to provide biocompatible, antimicrobial, and non-fouling surfaces. Accordingly, in another aspect, the invention provides devices and materials having a surface (i.e., one or more surfaces) to which have been applied (e.g., coated, covalently coupled, grafted to, grafted from, ionically associated, hydrophobically associated) one or more switchable polymers or switchable compounds of the invention. Representative devices and carriers that may be advantageously treated with a switchable polymer or switchable compound of the invention, modified to include a switchable polymer or switchable compound of the invention, or incorporates a switchable polymer or switchable compound of the invention include:

particle (e.g., nanoparticle) having a surface treated with, modified to include, or incorporates a switchable polymer or switchable compound of the invention;

drug carrier having a surface treated with, modified to include, or incorporates a switchable polymer or switchable compound of the invention;

non-viral gene delivery system having a surface treated with, modified to include, or incorporates a switchable polymer or switchable compound of the invention;

biosensor having a surface treated with, modified to include, or incorporates a switchable polymer or switchable compound of the invention;

devices for bioprocesses or bioseparations, such as membranes for microbial suspension, hormone separation, protein fractionation, cell separation, waste water treatment, oligosaccharide bioreactors, protein ultrafiltration, and diary processing having a surface treated with, modified to include, or incorporates a switchable polymer or switchable compound of the invention;

implantable sensor having a surface treated with, modified to include, or incorporates a switchable polymer or switchable compound of the invention;

subcutaneous sensor having a surface treated with, modified to include, or incorporates by a switchable polymer or switchable compound of the invention;

implant, such as a breast implant, cochlear implant, and dental implant having a surface treated with, modified to include, or incorporates a switchable polymer or switchable compound of the invention;

contact lens having a surface treated with, modified to include, or incorporates a switchable polymer or switchable compound of the invention;

tissue scaffold having a surface treated with, modified to include, or incorporates a switchable polymer or switchable compound of the invention;

implantable medical devices, such as an artificial joint, artificial heart valve, artificial blood vessel, pacemaker, left ventricular assist device (LVAD), artery graft, and stent having a surface treated with, modified to include, or incorporates a switchable polymer or switchable compound of the invention; and

medical devices, such as an ear drainage tube, feeding tube, glaucoma drainage tube, hydrocephalous shunt, keratoprosthesis, nerve guidance tube, urinary catheter, tissue adhesive, and x-ray guide having a surface treated with, modified to include, or incorporates by a switchable polymer or switchable compound of the invention.

Other representative substrates and surfaces that may be advantageously treated with a switchable polymer or switchable compound of the invention, modified to include a switchable polymer or switchable compound of the invention, or incorporates a switchable polymer or switchable compound of the invention include those that contact water, for example, pipes, heat exchangers, fishing nets, ship hulls, propellers, and marine structures (e.g., bridges and pilings).

Other representative substrates and surfaces that may be advantageously treated with a switchable polymer or switchable compound of the invention, modified to include a switchable polymer or switchable compound of the invention, or incorporates a switchable polymer or switchable compound of the invention include fabrics and such as in clothing (e.g., coats, shirts, pants, undergarments, including such as worn by hospital and military personnel), bedding (e.g., blankets, sheets, pillow cases, mattresses, and pillows), toweling, and wipes.

Other representative substrates and surfaces that may be advantageously treated with a switchable polymer or switchable compound of the invention, modified to include a switchable polymer or switchable compound of the invention, or incorporates a switchable polymer or switchable compound of the invention include working surfaces such as tabletops, desks, and countertops.

Methods for Using Switchable Surfaces

In a further aspect of the invention, methods for using switchable surfaces are provided.

In one embodiment, the invention provides a method for converting a surface of a substrate from a zwitterionic surface to a cationic surface. In the method, a surface of a substrate having a zwitterionic surface is contacted with acidic conditions to provide a cationic surface. In this embodiment, the zwitterionic surface comprises a layer of zwitterionic polymers of formula (I) or zwitterionic compounds of formula (III), and the cationic surface comprises a layer of cationic polymers of formula (II) or cationic compounds of formula (IV).

As described below, it will be appreciated that the conversion of a zwitterionic surface to a cationic surface can be substantially complete or, depending on the reaction conditions, controlled to provide a distribution of zwitterionic and cationic sites on the surfaces.

In the method, contacting the surface with acidic conditions includes contacting the surface with any one of a variety of acids in a variety of solvents. Suitable acids include mineral acids and organic acids. Representative acids include HCl, HBr, HCOOH, CH₃COOH, H₂SO₄, HSO₃F, HNO₃, H₃PO₄, HBF₄, HPF₆, H₃BO₃, CH₃SO₃H, CH₃CH₂SO₃H, C₆H₅SO₃H, CH₃C₆H₄SO₃H, CF₃SO₃H, CF₃COOH, citric acid, gluconic acid, lactic acid, oxalic acid, and tartaric acid.

Contacting the surface with acidic conditions includes contacting the surface with an acid in an aqueous or organic solvent.

In another embodiment, the invention provides a method for converting a surface of a substrate from a cationic surface to a zwitterionic surface. In the method, a surface of a substrate having a cationic surface is contacted with aqueous or basic conditions to provide a zwitterionic surface. In this embodiment, the cationic surface comprises a layer of cationic polymers of formula (II) or cationic compounds of formula (IV), and the zwitterionic surface comprises a layer of zwitterionic polymers of formula (I) or zwitterionic compounds of formula (III).

As described below, it will be appreciated that the conversion of a cationic surface to a zwitterionic surface can be substantially complete or, depending on the reaction conditions, controlled to provide a distribution of zwitterionic and cationic sites on the surfaces.

In the method, contacting the surface with aqueous or basic conditions comprises contacting the surface with water (pH≧7), aqueous buffers (pH≧7), or a basic organic solvent.

In the above methods, the zwitterionic surface is a non-fouling surface and the cationic surface is an antimicrobial surface.

In the methods described above, the surfaces can further include a functional molecule covalently coupled to one or more of the polymers or compounds of the layers.

The following is a description of a representative switchable polymers and switchable compounds of the invention, methods for making and using them, and surfaces modified to include them.

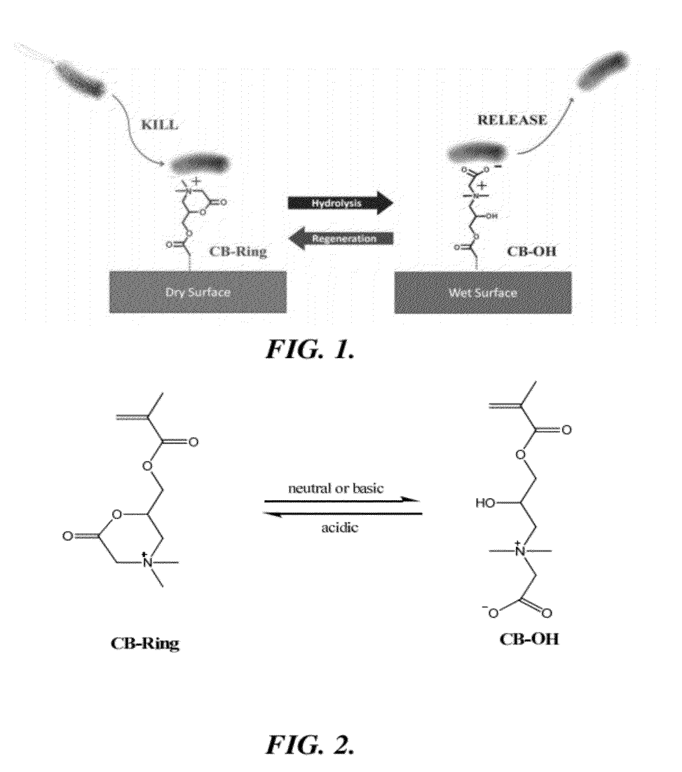

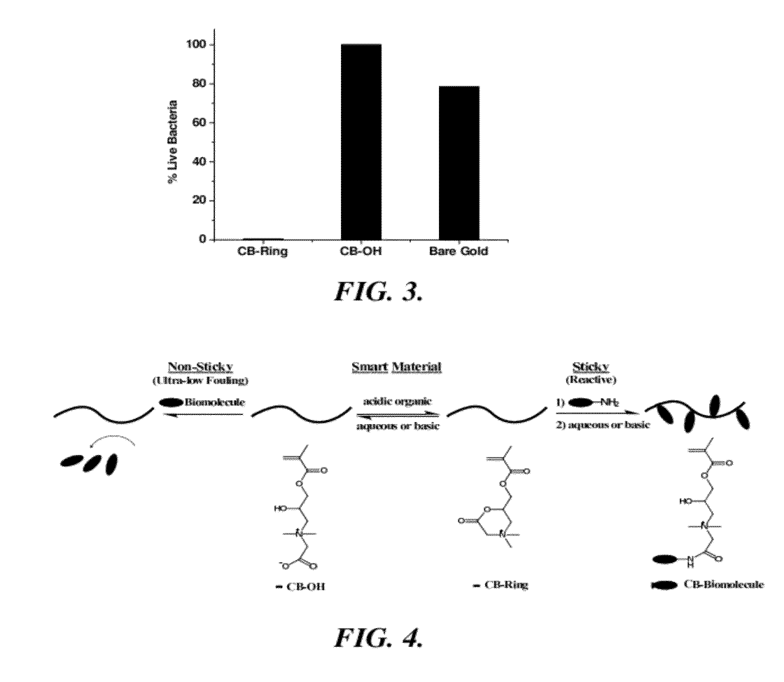

The coated surfaces reversibly switch between a cationic antimicrobial structure and zwitterionic non-fouling structure (FIG. 1). The surface is cationic (bacteria killing) when dry and zwitterionic (bacteria releasing) when wet. In one embodiment, this surface is based on a zwitterionic molecule, CB—OH (an open carboxylate form), which is in equilibrium with a cationic molecule, CB-Ring (a six-member lactone ring form) driven by basic or acidic environment (FIG. 2). Similar to other zwitterionic surfaces, the CB—OH surface is ultra-low fouling (fibrinogen binding<5 ng/cm²) from complex media. In addition, the CB-Ring surface can efficiently kill bacteria under dry conditions (FIG. 3). Additionally, the reversible surface can switch from cationic to zwitterionic form in a non-leaching (e.g., non-ethanol-leaching) process due to unique equilibrium chemistry.

CB—OH/CB-Ring surfaces can be prepared with varied parameters (e.g., film thickness, surface density, and switching conditions) that influence bacteria killing/releasing and surface recycling. Bactericidal activity works against several types of bacteria. Structure changes of this molecule can be monitored in situ at high sensitivity using surface-enhanced Raman scattering (SERS).

Additional aspects of the present disclosure are directed to coated surfaces on a quasi-3D gold nanostructure known to exhibit tunable SERS sensitivity. In this way, chemical signals (i.e., either ring or open structure of the material) can be collected to monitor the switchable properties of the material under different conditions, as well as structure change upon contact with bacteria (during the bacteria killing process).

The present invention also provides devices (e.g., filter membranes or fibers) having reversible switchable materials incorporated therein for repeated decontamination of biological threat agents. With switchable antimicrobial and non-fouling capabilities, these devices efficiently kill bacteria, release them, and recycle this kill-and-release function.

Integration of antimicrobial and non-fouling approaches and switching between two molecular structures having either antibiotic or non-fouling properties provides for materials and surfaces that can be directly applied to various porous fabrics used in a number of applications including, for example, solider uniforms, impacting C-WMD science.

“Non-sticky” and “sticky” properties are two highly desired material characteristics. The former refers to the ability to efficiently resist non-specific adsorption of biomolecules and microorganisms (i.e., non-fouling), while the latter enables the attachment of biomolecular recognition elements. The presence of both properties permits significant advancements in numerous applications such as biosensing, drug delivery, and tissue engineering. However, conventional wisdom prevents these two distinct properties from co-existing within a single material. For example, to achieve “sticky” properties, “non-sticky” materials must be either reacted to introduce functionalizable groups (e.g., carboxylate moieties) or reacted with coupling agents (e.g., carbodiimides) for conjugation to biomolecular recognition elements. Such chemistry has been applied to many materials, such as dextran, polyethylene glycol (PEG), and zwitterionic polymers. The present invention provides a “single” material containing a controllable “sticky” and “non-sticky” dual functionality.

In one aspect, the present invention a monomer, which can switch reversibly between an open-carboxylate form (CB—OH) and a lactone-ring form (CB-Ring) (e.g., a six-membered lactone ring) as shown in FIG. 4. This material can transition between these two equilibrium states driven by either acidic or basic conditions. The CB—OH, due to its zwitterionic structure is ultra-low fouling (“non-sticky”) while the CB-Ring, due to the lactone, is reactive (“sticky”) towards nucleophiles (e.g., amine moieties).

Ligand immobilization is illustrated in FIG. 4. CB—OH polymers can first be converted into the “sticky” state where ligand conjugation occurs via primary amine moieties. Unreacted CB-Ring groups can then be switched back into zwitterionic CB—OH resulting in a protein resistant background. A high throughput antibody array for early cancer diagnostics can be prepared using this technology.

The synthesis of CB—OH initially proceeds via the reaction of sarcosine tert-butyl ester with glycidyl methacrylate followed by the addition of methyl iodide to obtain the CB—OH t-butyl ester. Subsequent treatment with TFA to remove the protecting groups and neutralization via basic ion-exchange resins yields the final product, CB—OH, as a white powder after lyophilization (FIG. 12). The preparation and characterization of CB—OH is described in Example 1.

The open carboxylate form (CB—OH) has an equilibrium lactone-ring counterpart (CB-Ring) which forms in the presence of acidic media (dissolving CB—OH in TFA for 2 hours or overnight in a TFA/acetonitrile mixed solvent at 1/10 v/v). The resulting product, CB-Ring, was precipitated in ethyl ether. CB-Ring contains a characteristic ¹H NMR peak (m, 1H, CH₂═C(CH₃)COOCH₂CH(O—)CH₂—) at 5.53 ppm in TFA-d or 5.38 ppm in D₂O, which is absent in the zwitterionic state, and thus allowed the equilibrium kinetics to be quantified in different deuterated solvent environments (FIG. 5). It was found that in an acidic environment (i.e., TFA-d) CB—OH had a half-life of about 14 min and was fully converted into the CB-Ring structure within 2 hours. Going in the reverse direction using aqueous buffer at pH 7, the CB-Ring structure was quickly hydrolyzed with a half-life of about 4 min. Under basic conditions (i.e., pH 10) the half-life of CB-Ring became even shorter (<1 min) with complete conversion to the zwitterionic form in less than 6 min. The acidic or basic conditions necessary to drive the equilibrium in the corresponding directions are also indicated in FIG. 1.

The amino reactivity (i.e., the “sticky” characteristic) of the CB-Ring was then studied using a model molecule, benzylamine, in both aqueous and organic environments. The positively-charged lactone was efficiently conjugated to the amine group to provide CB—OH-benzyl conjugate. The control experiment, using the “non-sticky” zwitterionic form (CB—OH) as the starting material resulted in no conjugation. Referring to FIG. 5, the hydrolysis rate of CB-Ring strongly depends upon the condition used. Due to the basic character of benzylamine, any initial CB-Ring could undergo only conjugation or hydrolysis. By measuring the final concentration of CB—OH via HPLC and comparison to the control, a 5 molar excess of benzylamine with a 30 min reaction time resulted in efficiencies of 60% in pure water and 83% in acetonitrile. It is likely that the aqueous conjugating efficiency was lower due to the competition between aminolysis (to form the conjugate) and hydrolysis (to form CB—OH).

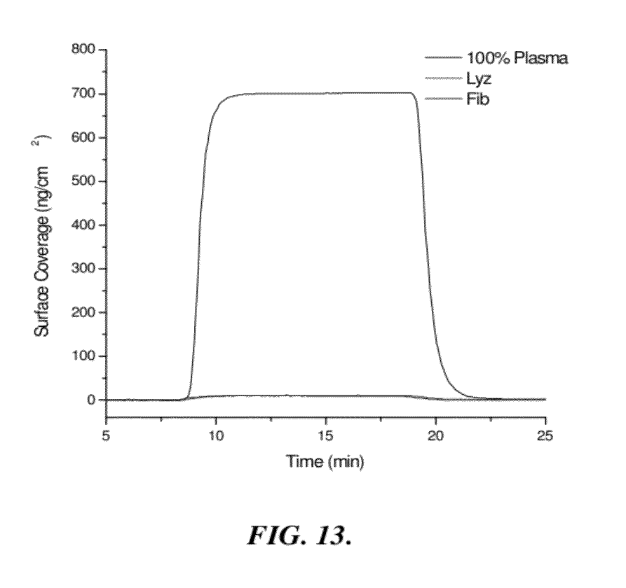

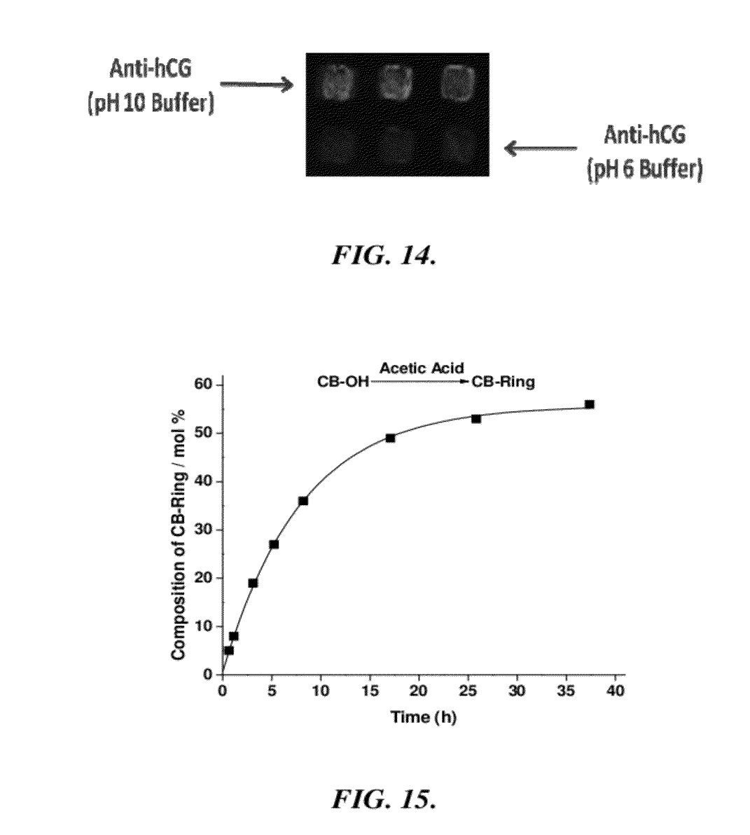

Previous zwitterionic materials have been found to be ultra-low fouling (“non-sticky”) by effectively resisting protein binding from undiluted human plasma and serum, in addition to adhesion from cells, bacteria, and other organisms. The term “ultra-low fouling” refers to low-fouling materials having fibrinogen adsorption levels less than 5 ng/cm², a level effectively inhibiting platelet adhesion which is necessary for blood compatibility. Although the introduction of an OH group into zwitterionic carboxybetaine provides CB—OH, the effect of the OH group is on the non-fouling properties is negligible. This was tested by using thin films of CB—OH (about 20 nm) formed via surface initiated atom transfer radical polymerization (SI-ATRP) from thiol initiators on gold substrates. SPR biosensors were then used to quantify non-specific protein binding. Single protein solutions of fibrinogen and lysozyme in PBS (1 mg/mL), as well as undiluted human plasma, were flowed over the CB—OH surface. Undetectable adsorption (<0.3 ng/cm²) was observed for the single proteins while 3.1±1.0 ng/cm² of protein fouling was detected for human plasma (see sensor-grams in FIG. 13). These results reveal that CB—OH was ultra-low fouling to all solutions analyzed (i.e., less than 5 ng/cm² of bound protein) and that this property was maintained despite modifying the original carboxybetaine compound. As a reference, a monolayer of protein binding results in a sensor response of 100-500 ng/cm².

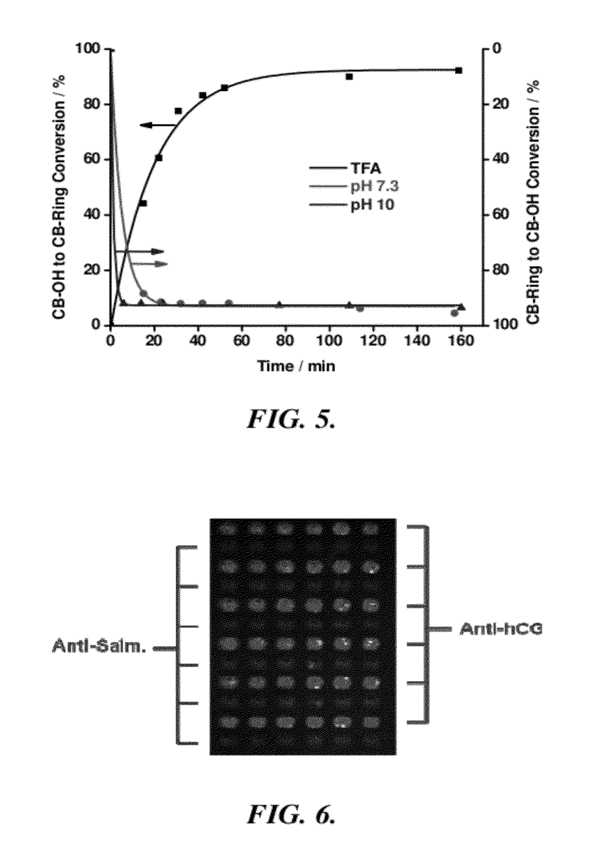

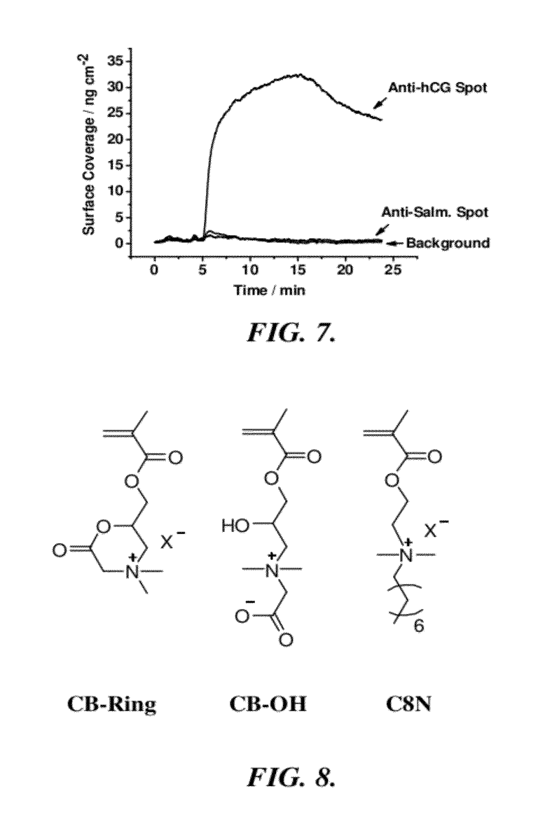

The “non-sticky” and “sticky” characteristics of the reversibly switchable polymers render these polymers useful in the creation of arrays (e.g., protein arrays). Using the same CB—OH substrates formed via SI-ATRP as above, a 10 min TFA treatment was used to form a sufficient amount of CB-Ring structures necessary for antibody immobilization. Two antibodies (anti-hCG and anti-Salm) were then contact printed onto the amine-reactive surface in pH 10 buffer resulting in a 6×12 array (FIG. 6). The control study showed immobilization in basic buffer (pH 10) was more efficient than slightly acidic buffer (pH 6) due to more deprotonated amines under basic conditions (FIG. 1). Subsequent hydrolysis of unreacted CB-Ring components using pH 9 buffer for 60 min switched the unspotted background from “sticky” into ultra-low fouling. Using an SPR imaging biosensor, significant specific detection of hCG was observed for anti-hCG spots while a zero response was observed for both the control antibody and the background (FIG. 7).

Conventional materials enable switching between hydrophobic and hydrophilic properties. Typical mechanisms for control include changes in light, pH, electric potential, temperature, and redox reactions. However, none of these switchable materials have achieved control over the two extreme properties as in the invention. The present invention provides “non-sticky” (resistance to protein adsorption from 100% blood plasma) and “sticky” (permanent covalent coupling) incompatible properties were controlled using a single material.

Switching Reversibly Between Attacking and Defending Against Bacteria

There are two major approaches to prevent the microbial colonization and biofilm formation on a surface. The active approach is to “attack” by killing bacteria with a wide range of antimicrobial materials or drugs including, but not limited to, cationic polymers, antimicrobial peptides, antibiotics, silver ions, nitrous oxide. However, poor biocompatibility or drug resistance is of great concern. The passive approach is to “defend” by resisting bacteria using non-fouling coatings, such as polyethylene glycol (PEG) and zwitterionic, and their derivative coatings. However, once bacteria are attached on a surface, there is no mechanism to kill them. Ideally, a surface is able to perform the inter-switchable functions each at a time, such as to kill bacteria attached, release killed microbes and either return to the initial killing state or maintain the final non-fouling state. Cationic ester surfaces provide a “kill and release” strategy based on a cationic ester. The cationic ester surface was able to kill bacteria attached and release dead microbes upon the hydrolysis of ester groups resulting in a non-fouling zwitterionic surface. Unfortunately, this is a one-time action and the surface cannot be regenerated back to its original form because the hydrolysis of ester groups is not reversible.

The present invention provides a single material that switches reversibly and easily between attacking (i.e., cationic) and defending (i.e., zwitterionic) forms. The single material is a polymer capable of switching repeatedly between its two equilibrium states: a cationic N,N-dimethyl-2-morpholinone (CB-Ring) and a zwitterionic carboxy betaine (CB—OH), respectively (FIG. 1). The CB-Ring kills over 99.9% of Escherichia coli K12 (E. coli K12) attached on it under dry conditions. In neutral or basic aqueous environments, CB-Ring is hydrolyzed to CB—OH, which releases dead bacteria and at the same time resists bacteria adhesion in the aqueous media. CB—OH can finally be converted back to CB-Ring under acidic conditions regenerating the bacteria killing function. As compared with the known “one-time switch” surface, switching of the polymer of the invention is fully reversible and triggered under practical conditions such as regenerating the bacteria-killing function via weak acid (e.g., acetic acid) and shifting to bacteria releasing and resisting functions immediately in the physiological environment.

The bacteria attacking function of CB-Ring is based on its structural characteristics, the cationic, quaternary amine group and its similarity to 2-morpholinone. It is well known that quaternary amine compounds are able to efficiently kill a variety of microorganisms both on surfaces and in bulk solution. In addition, 2-morpholinone derivatives (with unsubstituted nitrogen atoms) have shown pronounced activity against bacterial and fungal species. CB—OH monomer (FIG. 8) was synthesized and its polymer thin film (film thickness=26.6±0.6 nm) was developed through surface-initiated atom-transfer radical polymerization from thiol initiators on gold substrates. Because CB—OH has a half-life of 14 min in trifluoroacetic acid (TFA) before being fully converted to CB-Ring, the CB—OH surface was dipped in TFA for 20 min to generate sufficient amount of CB-Ring on the surface followed by repeated washes with acetonitrile.

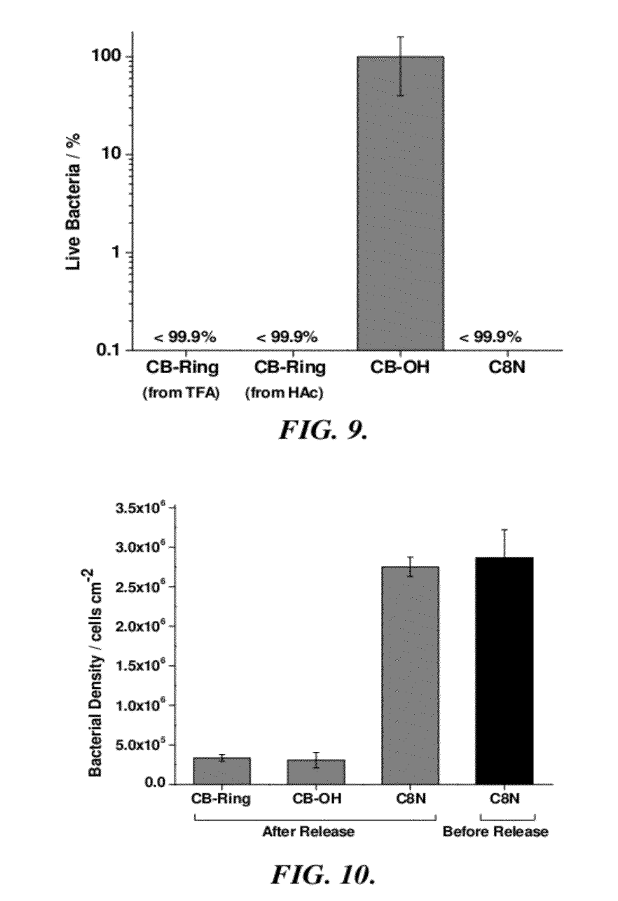

The bactericidal activity of CB-Ring surface was tested on E. coli K12, using an established assay. A typical quaternary amine surface known for its bactericidal ability (C8N), (film thickness=26.6±0.5 nm) and the zwitterionic CB—OH surface were used as the positive and negative controls, respectively (FIG. 8). It was observed that cationic CB-Ring generated from TFA killed more than 99.9% of the bacteria sprayed on its surface in 1 h under dry conditions, similar to the cationic C8N surface (FIG. 9). CB-Ring generated from acetic acid (HAc) had equally effective bactericidal activity. CB—OH being zwitterionic had no bactericidal activity at its dry state (100% live bacteria in FIG. 8), showing roughly the same amount of live bacteria similar to a bare gold surface.

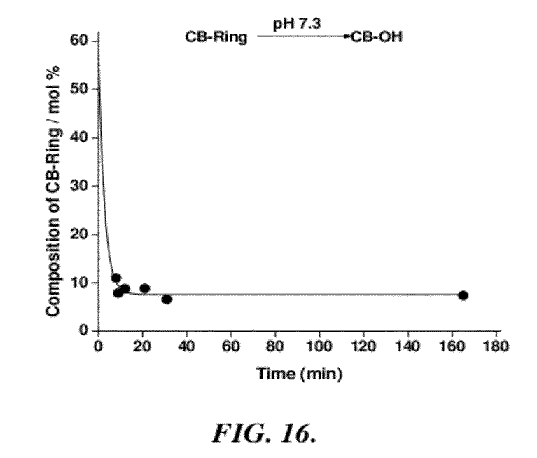

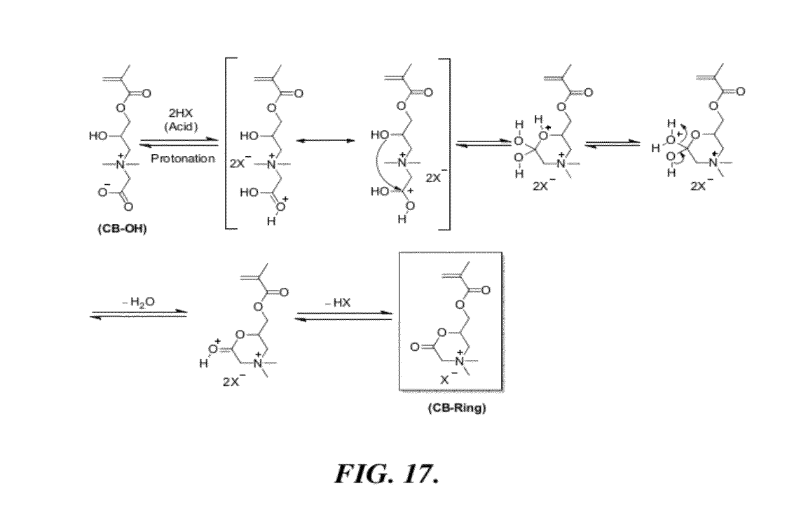

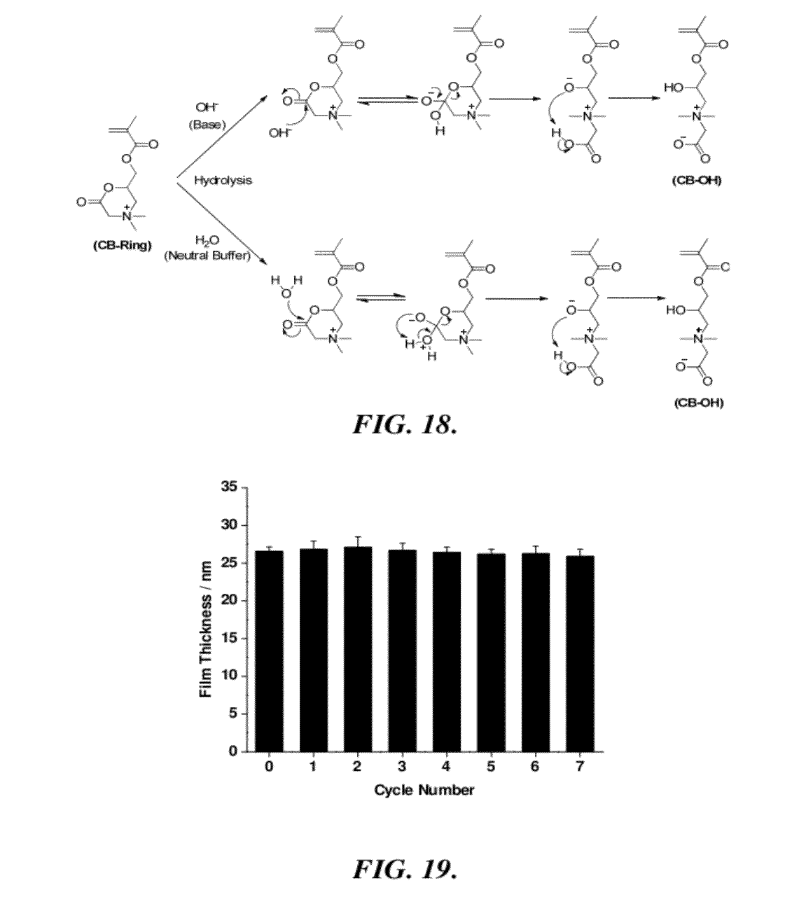

CB-Ring can be regenerated by two methods: TFA or HAc. HAc effectively converted CB—OH to CB-Ring. As a weak acid, HAc does not erode away the gold layer on our substrates and can be used to repeatedly treat the surface without destabilizing the polymer coating. The conversion of CB—OH to CB-Ring was examined by dissolving CB—OH monomer into [D]HAc at a concentration of 0.2 M and the characteristic ¹H NMR peaks of CB—OH and CB-Ring (FIG. 15) were compared. It was found that 20 hours [D]HAc treatment led to 50% CB—OH converted to CB-Ring. The equilibrated product at 40 h was precipitated in ethyl ether, vacuum dried, and placed into pH=7.3 buffer made from 200 mM Na₂CO₃ in D₂O titrated with DCl. ¹H NMR indicated a fast hydrolysis of CB-Ring back to CB—OH with a nearly full conversion in less than 10 min (FIG. 16). A mechanism for the equilibrium between CB-Ring and CB—OH is illustrated in FIG. 17. The conversion from CB—OH to CB-ring is catalyzed by the protonation of the carboxylate group leading to an intramolecular Fischer esterification involving the neighboring hydroxyl group. A strong acid is the ideal catalyst for the reaction as is the case for TFA which gave an almost complete conversion to CB-ring in 1 h; while HAc, a weak acid, yielded a 50% conversion after 20 h. The bactericidal activity of CB-Ring surface generated by HAc treatment for 20 h. was not fully converted to CB-Ring. However, the surface was as efficient as the TFA generated CB-Ring surface by killing E. coli K12 greater than 99.9% in the dry condition (FIG. 9). Furthermore, the polymer film stability was tested by switching the surface between CB—OH and CB-Ring repeatedly, through various cycles of 20 h HAc and 2 h phosphate buffered saline (PBS) treatment (CB—OH→CB-Ring and CB-Ring→CB—OH, respectively). No obvious film deterioration was found as indicated by a constant film thickness of CB—OH over 7 repeated cycles (FIG. 19).

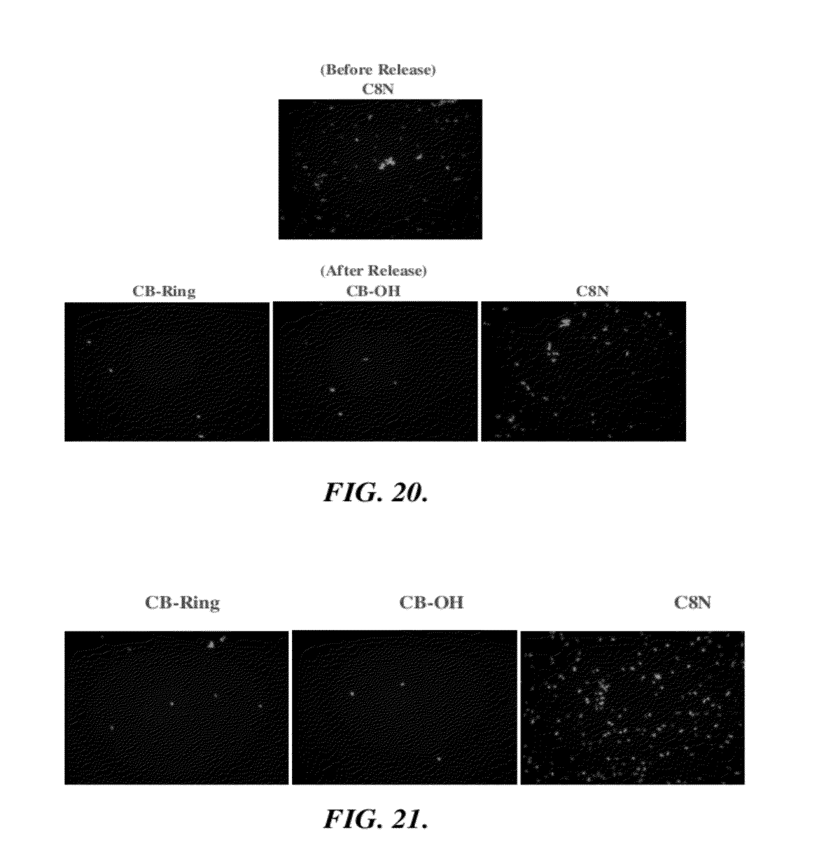

For the bacteria defending function, the ability of the material to release the previously attached/dried (killed) bacteria in wet conditions and to further resist bacteria attachment was evaluated. To test the bacteria releasing capability, surfaces previously sprayed with E. coli K12 and dried for 15 min, were gently shaken in PBS for 1 h. Bacteria were stained with FM® 1-43 and their surface density before and after the shaking (releasing) were monitored under a fluorescence microscope (quantitative data in FIG. 10; representative images in FIG. 20). It was found that CB-Ring surface was able to release 90% previously attached/dried bacteria, similar to CB—OH. It was expected that upon the contact with PBS, cationic CB-Ring was quickly converted to CB—OH within minutes (FIG. 17). As shown previously, CB—OH surface was ultra-low fouling in resisting fibrinogen and undiluted human plasma, with less than 5 ng cm⁻² adsorbed proteins. As a reference, a monolayer of protein binding results in a surface coverage of 100-500 ng cm⁻². The non-fouling nature of CB—OH accounts for the efficient release of the attached/dried bacteria. In contrast, the permanent cationic C8N exhibited no observable bacteria release (p>0.6, One-Way ANOVA) (FIG. 10).

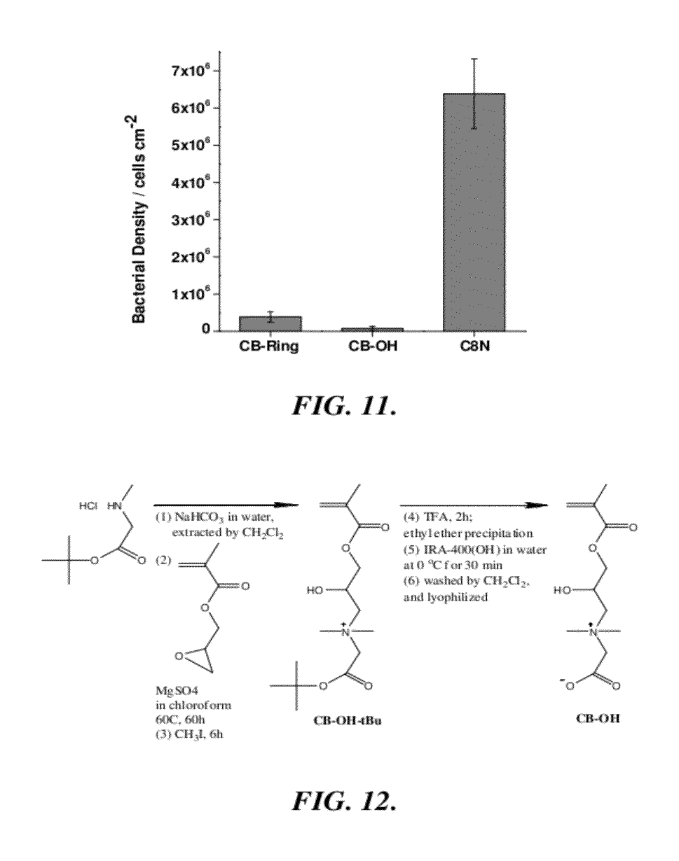

To test the bacteria resistance in wet conditions, surfaces were incubated with E. coli K12 solution (10⁸ cells mL⁻¹ in PBS) for 0.5 h, and shaken in fresh PBS for 1 h to remove unbounded bacteria. Bacteria remained on the surfaces were stained with FM® 1-43 for visualization. It was found that the surface with its initial state at both CB-Ring and CB—OH effectively resisted the bacteria adhesion, while C8N had large amounts of bacteria adhesion due to its cationic and hydrophobic nature (quantitative data in FIG. 11; representative images in FIG. 21). An established ultra-low fouling zwitterionic surface made from polycarboxy betaine (PCBMA) was tested as a negative control (film thickness=32.2±0.3 nm) and showed an extremely low bacteria coverage of 6.4±3.2×10⁴ cells cm⁻². The bacteria surface density for CB—OH was 7.8±5.8×10⁴ cells cm⁻², which was equally as low as the PCBMA control (p>0.6, One-Way ANOVA). As a reference, a single observable bacterial under the field of the microscope in our method set-up represented a bacteria density of 3.2×10⁴ cells cm⁻².

The bacteria attacking ability of CB-Ring was examined after one cycle of switch. CB-Ring surface was sprayed with E. coli K12, dried for 1 h, and shaken in PBS for another hour to release the killed bacteria. The resulting CB—OH surface was treated with HAc for 20 h to regenerate CB-Ring. The resulting CB-Ring surface was tested for bactericidal activity, and was found to be as effective as in its previous cycle (i.e., kill E. coli K12 over 99.9%).

The following examples are provided for the purpose of illustrating, not limiting, the invention.

### EXAMPLES

### Example 1

The Preparation and Characteristics of a Representative Switchable Polymer

In this example, the preparation and characterization of a representative switchable polymer of the invention is described.

N-(2-tert-Butoxy-2-oxoethyl)-2-hydroxy-3-(methacryloyloxy)-N,N-dimethylpropan-1-aminium iodide (CB—OH-tBu in FIG. 12). 9.37g (1N) of sarcosine tert-butyl ester hydrochloride (Sigma, Milwaukee) was neutralized by 5 g (1.2N) of sodium bicarbonate in 60 ml water. The resulting sarcosine tert-butyl ester was extracted with 60 ml dichloromethane and obtained as 7.4 g (1N) of liquid after solvent evaporation. The purified sarcosine tert-butyl ester was reacted with 10.39 ml (1.5N) glycidyl methacrylate (TCI America, Portland) in the presence of 0.4 g magnesium sulfate and 20 ml of dichloromethane at 60° C. for 60 h under N₂ protection. After the reaction, insoluble magnesium sulfate was removed by filtration and the filtrate was mixed with 140 ml methyl iodide (methyl iodide/filtrate volume ratio 4/1). As the methylation reaction proceeded white CB—OH-tBu crystals formed. CB—OH-tBu was further washed with ethyl ether and vacuum-dried before use. (Yield 54%) ¹H NMR (chloroform-d) δ (ppm): 1.44 (s, 9H, —OC(CH₃)₃), 1.89 (s, 3H,CH₂═C(CH₃)COO—), 3.62 (d, J=6 Hz, 6H, —CH₂N(CH₃)₂CH₂COO—), 3.85-4.04 (m, 2H, CH₂═C(CH₃)COOCH₂CH(OH)CH₂N(CH₃)₂CH₂—), 4.18 (d, J=4.8 Hz, 2H, —CH₂N(CH₃)₂CH₂COO—), 4.47-4.58 (m, 2H, CH₂═C(CH₃)COOCH₂CH(OH)—), 4.62-4.68 (m, 1H, CH₂═C(CH₃)COOCH₂CH(OH)CH₂—), 5.58 and 6.14 (s, 2H, CH₂═C(CH₃)COO—). ¹³C NMR (chloroform-d) δ (ppm): 18.53 (CH₂═C(CH₃)COO—), 28.15 (—OC(CH₃)₃), 54.41 (d, J=78 Hz, —CH₂N(CH₃)₂CH₂—), 62.84 (—CH₂N(CH₃)₂CH₂COO—), 63.69 (—CH₂CH(OH)CH₂—), 65.45 (—CH₂CH(OH)CH₂N(CH₃)₂CH₂—), 66.03 (—COOCH₂CH(OH)CH₂—), 85.62 (—OC(CH₃)₃), 127.08 (CH₂═C(CH₃)COO—), 135.59 (CH₂═C(CH₃)COO—), 163.56 (CH₂═C(CH₃)COO—), 167.17 (—COOC(CH₃)₃).

2-((2-Hydroxy-3-(methacryloyloxy)propyl)dimethylammonio)acetate (CB—OH in FIGS. 2 and 12). CB—OH-tBu crystals were dissolved in trifluoroacetic acid (TFA) (0.5 g CB—OH-tBu/mL TFA) for 2 h to fully deprotect the tBu groups. The resulting product was precipitated in ethyl ether, re-dissolved in water, and stirred with Amberlite IRA-400(OH) ion-exchange resin (Sigma, Milwaukee) at 0° C. for 30 min. The filtrate containing CB—OH was washed with methylene chloride three times and lyophilized as a white powder. (Yield 96%) ¹H NMR (D₂O) δ (ppm): 1.87 (s, 3H, CH₂═C(CH₃)COO—), 3.25 (d, J=8.5 Hz, 6H, —CH₂N(CH₃)₂CH₂COO—), 3.74 (d, J=4.5 Hz, 2H, CH₂═C(CH₃)COOCH₂CH(OH)CH₂N(CH₃)₂CH₂—), 3.94 (m, 2H, —CH₂N(CH₃)₂CH₂COO—), 4.14 (m, 2H, CH₂═C(CH₃)COOCH₂CH(OH)—), 4.47 (m, 1H, CH₂═C(CH₃)COOCH₂CH(OH)CH₂—), 5.68 and 6.10 (s, 2H, CH₂═C(CH₃)COO—). ¹³C NMR (D₂O) δ (ppm): 17.42 (CH₂═C(CH₃)COO—), 52.82 (d, J=12.5 Hz, —CH₂N(CH₃)₂CH₂—), 64.00 (—CH₂CH(OH)CH₂—), 64.61 CH₂CH(OH)CH₂N(CH₃)₂CH₂—), 65.01 (—COOCH₂CH(OH)CH₂—), 66.53 (—CH₂N(CH₃)₂CH₂COO⁻), 127.50 (CH₂═C(CH₃)COO—), 135.37 (CH₂═C(CH₃)COO—), 168.88 (CH₂═C(CH₃)COO— and —CH₂COO⁻). To rule out the presence of undesired TFA esters which could form as a result of reacting with the hydroxyl groups of CB—OH, ¹⁹F NMR studies were conducted. Upon testing CB—OH in D₂O, the resulting spectra lacked a peak at −76.68 ppm, indicating the absence of both TFA⁻ counter ions and TFA esters. Ion Trap Mass Spectrometer (IT-MS, Bruker Esquire) further confirmed the result by giving a m/z of 246.1 for the protonated form of CB—OH (calculated MW for C₁₁H₁₉NO₅+H was 246.3). The fragmentation product revealed a m/z of 143.1.

2-(Methacryloyloxymethyl)-4,4-dimethyl-6-oxomorpholin-4-ium (CB-Ring in FIG. 2). CB-Ring was obtained by dissolving CB—OH in TFA for 1.5 h or in TFA/acetonitrile mixed solvent ( 1/10 v/v) overnight. ¹H NMR (TFA-d) δ (ppm): 2.57 (s, 3H, CH₂═C(CH₃)COO—), 4.14 (s, 6H, —CH₂N(CH₃)₂CH₂COO—), 4.53 and 4.76 (m, 2H, CH₂═C(CH₃)COOCH₂CH(OCO—)CH₂N(CH₃)₂CH₂—), 5.01-5.15 (m, 2H, —CH₂N(CH₃)₂CH₂COO—), 5.20-5.34 (m, 2H, CH₂═C(CH₃)COOCH₂CH(OCO)—), 6.05 (m, 1H, CH₂═C(CH₃)COOCH₂CH(OCO—)CH₂—), 6.43 and 6.87 (s, 2H, CH₂═C(CH₃)COO—). ¹³C NMR (TFA-d) δ (ppm): 16.56 (CH₂═C(CH₃)COO—), 52.13, 57.06, 58.79, 61.14, 63.17, 73.08 (d, —CH₂N(CH₃)₂CH₂—; —CH(OCO)CH₂N(CH₃)₂CH₂—; CH₂═C(CH₃)COOCH₂—; CH₂═C(CH₃)COOCH₂CH(OCO)CH₂—; —CH₂N(CH₃)₂CH₂COO—), 129.65 (CH₂═C(CH₃)COO—), 134.73 (CH₂═C(CH₃)COO—), 162.82 (CH₂═C(CH₃)COO—), 170.21 (—N(CH₃)₂CH₂COO—). IT-MS showed a m/z of 228.1 (calculated MW for C₁₁H₁₈NO₄+ was 228.3). The fragmentation products resulted in m/z values of 200.0 and 84.2.

Kinetic study between CB—OH and CB-Ring. The equilibrium conversion between the CB—OH and CB-Ring was monitored by ¹H NMR at various time points. To monitor the formation of CB-Ring, CB—OH powder was initially dissolved in TFA-d at a concentration of 0.2 M. The conversion from CB-Ring to CB—OH was studied by dissolving CB-Ring in either pH 7.3 or pH 10 buffer at a concentration of 0.2 M. These two buffers were made from 200 mM Na₂CO₃ in D₂O and were titrated with DCl. Conversion was calculated based on the ratio of the characteristic peak for CB-Ring (1H, CH₂═C(CH₃)COOCH₂CH(O—)CH₂—) and a common peak of both structures (6H, —CH₂N(CH₃)₂CH₂—). In FIG. 5, the final conversion within 160 min was about 93% for all three conditions investigated. Because the integration error for ¹H NMR can be as large as 10%, a value of 93% essentially represented an almost full conversion.

“Sticky” CB-Ring. The sticky or amino reactive property of CB-Ring was studied via the reaction with a model molecule, benzylamine. Briefly, 7 mg CB-Ring was reacted with 17 μl benzylamine (5N) for 30 min in 100 μl of either acetonitrile or water. The resulting mixture was analyzed via liquid chromatography-mass spectrometry (HPLC-MS). HPLC-MS was set up by integrating a Hewlett Packard 1100 liquid chromatograph system with a Bruker Esquire IT-MS. The HPLC unit was equipped with a reverse-phase column (Agilent Zorbax SB-C18, 2.1×100 mm, 3.5 μm) and a UV detector (wavelength of 210 nm). A gradient mobile phase was applied at a flow rate of 0.2 ml/min (A: H₂O with 5% B; B: acetonitrile with 1% acetic acid). The linear gradient was set with 0% B at 0 min, 50% B at 15 min, and 100% B at 16.5 min. CB—OH-benzyl conjugate was isolated and identified with a m/z of 335.4 (calculated MW for C₁₈H₂₇N₂O₄+ is 335.4). The fragmentation product of CB—OH revealed a m/z of 143.1. Control CB—OH showed no formation of CB—OH-benzyl conjugates. To quantify the conjugation efficiency under the tested conditions, a HPLC calibration curve for CB—OH was developed by dissolving known amounts of CB-Ring in 116 μl phosphate buffer (1M, pH 7) for 1 h and analyzing the resulting mixture for the peak areas (at 4 min) for the fully converted CB—OH form. Because CB-Ring can only be conjugated to benzylamine or hydrolyzed to CB—OH, the conjugation efficiency was estimated by quantifying the amount of CB—OH remaining relative to the control experiment.

Preparation of CB—OH polymer brush. Gold-coated SPR sensor chips (glass coated with 2 nm of titanium followed by 48 nm of gold) were rinsed with ethanol and Millipore water, dried with filtered air, and placed in a UV chamber for 20 min. The chips were then rinsed with water and ethanol and then incubated with a 0.1 mM ethanolic solution of mercaptoundecyl bromoisobutyrate overnight. These substrates were further rinsed with ethanol and tetrahydrofuran before use. CB—OH polymer brushes were synthesized via surface initiated atom transfer radical polymerization (ATRP). Here, 21.4 mg of copper(I) bromide (Sigma, Milwaukee), 46.28 mg of 2,2-bipyridine (BPY, Sigma, Milwaukee), and two SPR chips were placed in a small tube reactor under nitrogen protection. 1.5 mL of nitrogen-purged solvent (DMF/water 3/1) was used to dissolved 225 mg of CB—OH and was then transferred to the reactor. After 3 h, the chips were removed, rinsed with ethanol and water, and then stored in PBS. The polymer film thickness was measured by ellipsometry as 19.6±0.8 nm (from three measurements) and the refractive index of CB—OH polymer film was estimated to be 1.46 (α-SE^{S}, J.A. Woollam Co., Inc.).

Measurement of protein adsorption by a SPR sensor. A custom-built four-channel SPR sensor was used to quantify the absolute value of protein adsorption on CB—OH polymer brushes. PBS buffer at 50 μl/min and 25° C. was first used to obtain a stable baseline. A 1 mg/ml protein solution was then injected for 10 minutes followed by buffer to remove any loosely bound proteins. Non-specific adsorption of fibrinogen (from bovine plasma, Sigma) and lysozyme (from chicken egg white, Sigma) was calculated as the change in wavelength between the baselines before and after protein injection. For bare-gold SPR substrates, a 1 nm wavelength shift starting at a resonant wavelength of 750 nm represents 17 ng/cm² of absorbed proteins. The detection limit, based on 3 standard deviations of baseline noise, is about 0.2 ng/cm². For polymer films on SPR substrates, the increase in the distance between the binding event and the SPR active gold surface lowers the sensitivity which can be accounted for with a theoretical model. Using the refractive index of CB—OH polymer brushes and the wavelength shift (relative to bare gold substrates) caused by the polymer film, a calibration factor can be predicted. For example, a 19.6 nm CB—OH polymer film gives a 95 nm wavelength shift and has a calibration factor of 1.28. Thus, for this specific CB—OH coated chip, a 1 nm shift in resonant wavelength represents 21.8 ng/cm² of protein coverage.

Antibody arrays on CB—OH coated substrates sensor. The CB—OH films formed by SI-ATRP were first converted into the polymeric ring structure (CB-Ring) which enabled subsequent reactivity with primary amines. These substrates were then used to generate antibody microarrays with a Spotbot2 personal microarrayer (TeleChem International, Inc.). Monoclonal antibody to the β-subunit of human Chorionic Bonadotropin (anti-hCG, Scripps Laboratories) and polyclonal antibody to Salmonella (anti-Salm, Meridian Life Science, Inc.) were then spotted using a SMP8 Stealth micro-contact printing pin. This resulted in an array of proteins each with a diameter of about 265 μm. Antibodies were spotted at a concentration of 1 mg/mL in 100 mM sodium carbonate buffer (pH 6, 10, and 11) and allowed to react for one hour at about 70% humidity and room temperature. The substrates were then immediately submerged in a solution consisting of 10 mM boric acid and 300 mM sodium chloride (BANa pH 9.0) for one hour. BANa was used to completely hydrolyze the “sticky” state (CB-Ring) back into the ultra-low fouling zwitterionic state (CB—OH) in addition to removing non-covalently attached antibodies. The chip was then removed, rinsed with water, dried, and mounted onto a self-referencing SPR imaging biosensor containing polarization contrast. This instrument has a response resolution better than 10⁻⁶ refractive index units (RIU) and an operating range of 0.011 RIU thus enabling highly sensitive detection.

Biomarker detection in a SPR imagining sensor. PBS was used as the running buffer to demonstrate the detection abilities of the microarray platform. The flow rate for all steps in this protocol was 50 μL/min. After establishing a stable baseline with running buffer, 0.4% and 0.8% NaCl solutions in PBS (w/w) were flowed sequentially for 3 minutes each followed by PBS. Each of two flow-channels contained a total of 72 antibody spots allowing for 144 simultaneous and independent measurements. The salt-step RI calibration was necessary to normalize the slight variations in the sensor response of the background (CB—OH) and each of the 144 individual protein spots. Detection was then performed by flowing a solution of hCG (10 μg/mL in PBS) for 10 minutes followed by a 10-min PBS wash. Following the experiment, the chip was removed and mounted onto a spectroscopic SPR sensor using a clean area which was not previously used for printing. The same salt-step RI calibration was completed and the result was used, along with the calibration factor, to convert the RI response values to protein surface coverage.

### Example 2

Antimicrobial Effect of a Representative Switchable Polymer

In this example, the antimicrobial effect of a representative switchable polymer of the invention is described.

Attacking and defending against bacteria. A switchable polymer surface has two reversibly switchable equilibrium states, CB-Ring and CB—OH. CB-Ring will kill bacteria upon contact under dry conditions, while CB—OH will release the previously attached/dead bacteria and further resist bacteria adhesion under wet conditions.

Preparation of the materials and the polymer surfaces. CB—OH monomers were prepared as described herein. CB—OH polymer brush was grown on a gold coated substrate as described in Example 1.

Kinetic study between CB—OH and CB-Ring in acetic acid/pH=7.3 buffer. ¹H NMR was used to monitor the equilibrium conversion between the CB—OH and CB-Ring. CB—OH monomer was initially dissolved in [D]acetic acid at a concentration of 0.2 M. At various time points, the spectra were collected, and composition for the formed CB-Ring was calculated based on the ratio of the characteristic peaks for CB-Ring and CB—OH (FIG. 16). The equilibrated product (a mixture of CB-Ring and CB—OH) in [D]acetic acid was precipitated in ethyl ether, vacuum dried, and placed into pH=7.3 buffer made from 200 mM Na₂CO₃ in D₂O titrated with DCl. CB-Ring was hydrolyzed to CB—OH at this condition and the composition for the CB-Ring at various time points were measured (FIG. 16).

Bacteria killing (bactericidal activity) test. E. coli K12 was cultured overnight at 37° C. on Luria-Bertani (LB) agar plates. One colony was picked and cultured in 25 mL of LB medium (20 g/L) for 16 hours at 37° C. with a shaking speed at 200 rpm (Boekel grant ORS200 shaking water bath, Grants instruments (Cambridge) Ltd, UK). The resulting culture was used to inoculate a second culture in 200 mL of LB medium until an optical density of 0.8 at 600 nm was reached. The bacteria were collected by centrifugation at 8,000×g for 10 min at 4° C., washed three times with sterile PBS (pH 7.4) and finally suspended in water to get a final concentration of 10⁷ cells mL⁻¹. The bacteria suspension was sprayed onto the polymer surfaces using a chromatography sprayer (VWR scientific) at a spray rate of about 10 mL min⁻¹. The samples were dried at room temperature for 1 hour, transferred to sterile Petri dishes and covered with a layer of LB medium containing 0.8% agar (previously autoclaved, and cooled to 37° C.). After overnight incubation at 37° C., bacterial colonies became visible and were counted.

Bacteria releasing test. E. coli K12 was sprayed onto polymer surfaces as described in the bacterial killing test section, and allowed to dry for 15 min. The samples were then incubated with 5 ml of fresh PBS in a 6-well plate with gentle shaking for 1 h (to release the bacteria previously attached/dried on the surface). Bacteria remained on the polymer surfaces were stained with FM® 1-43 (Invitrogen, Carlsbad, Calif., US) according to the manufacture protocol. The number of bacteria cells was determined with a CCD-CoolSNAP camera (Roper scientific, Inc., USA) mounted on Nikon Eclipse 80i with 100× lens through a FITC filter. Under the experimental set-up, one bacterial cell appeared in a recorded picture represented a bacteria density of 3.2×10⁴ cells cm⁻².

Bacteria resisting test. A 5 mL suspension of E. coli K12 (10⁸ cells mL⁻¹ in PBS) was incubated with the polymer surfaces in a 6-well plate. After incubation for half an hour, the bacteria medium was replaced by fresh PBS with gentle shaking for 1 h (to remove unbound bacteria). The bacteria attached on the polymer surfaces were stained and counted according to the procedure described in the bacterial releasing test section.

While illustrative embodiments have been illustrated and described, it will be appreciated that various changes can be made therein without departing from the spirit and scope of the invention.

1. A polymer having the formula: 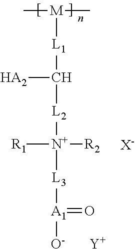
wherein
M is a monomeric repeating unit;
L₁ is a first linker that covalently couples the methine carbon to the repeating unit;
A₂ is O, NR₃, or S, wherein R₃ is selected from the group consisting of hydrogen, C1-C6 alkyl, and C6-C12 aryl;
R₁ and R₂ are independently selected from the group consisting of hydrogen, C1-C6 alkyl, and C6-C12 aryl, or taken together with the nitrogen to which they are attached form a cationic center;
L₂ is a second linker that covalently couples the methine carbon to the cationic center;
A₁(=O)—O⁻ is an anionic center, wherein A₁ is selected from the group consisting of C, Si, SO, and POH;
L₃ is a third linker that covalently couples the cationic center to the anionic center;
X⁻ is a counter ion associated with the cationic center;
Y⁺ is a counter ion associated with the anionic center; and
n is an integer from 1 to about 10,000.

2. The polymer of claim 1, wherein the monomeric repeating unit is selected from the group consisting of monomeric repeating units for polyesters, polyamides, poly(amino acids), polyimides, polycarbonates, polysiloxanes, polyurethanes, polyphosphazenes, acrylic polymers, amino resins, epoxy resins, phenolic resins, and alkyd resins.

3. The polymer of claim 1, wherein the monomeric repeating unit is —C(R₄)(R₅)—C*(R₆)—, wherein R₄, R₅, and R₆ are independently selected from the group consisting of hydrogen, fluorine, substituted and unsubstituted C1-C6 alkyl, and substituted and unsubstituted C6-C12 aryl, and wherein * represents the point of attachment to L₁.

4. The polymer of claim 1, wherein L₁ is selected from the group consisting of —C(═O)O—(CH₂)ₙ— and —C(═O)NH—(CH₂)ₙ—, wherein n is an integer from 1 to 20;

5. The polymer of claim 1, wherein L₂ is —(CH₂)ₙ—, wherein n is an integer from 0 to 3, and L₃ is —(CH₂)ₘ—, wherein m is an integer from 1 to 4, wherein n+m≦4.

6. The polymer of claim 1, wherein R₁ and R₂ taken together with the nitrogen to which they are attached form cationic center selected from the group consisting of quaternary ammonium, imidazolium, triazolium, pyridinium, and morpholinium.

7. A polymer having the formula: 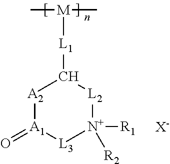
wherein
M is a monomeric repeating unit;
L₁ is a first linker that covalently couples the methine carbon to the repeating unit;
A₂ is O, NR₃, or S, wherein R₃ is selected from the group consisting of hydrogen, C1-C6 alkyl, and C6-C12 aryl;
R₁ and R₂ are independently selected from the group consisting of hydrogen, C1-C6 alkyl, and C6-C12 aryl, or taken together with the nitrogen to which they are attached form a cationic center;
L₂ is a second linker that covalently couples the methine carbon to the cationic center;
L₃ is a third linker that covalently couples the cationic center to A₁;
A₁ is selected from the group consisting of C, Si, SO, and POH;
X⁻ is a counter ion associated with the cationic center;
Y⁺ is a counter ion associated with the anionic center; and
n is an integer from 1 to about 10,000.

8. A surface of a substrate, wherein the surface comprises a layer of the polymers of claim 1.

9. A surface of a substrate, wherein the surface comprises a layer of the polymers of claim 7.

10. A surface of a substrate, wherein the surface comprises a layer of first and second polymers,
wherein the first polymers have the formula 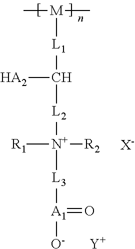
wherein
M is a monomeric repeating unit;
L₁ is a first linker that covalently couples the methine carbon to the repeating unit;
A₂ is O, NR₃, or S, wherein R₃ is selected from the group consisting of hydrogen, C1-C6 alkyl, and C6-C12 aryl;
R₁ and R₂ are independently selected from the group consisting of hydrogen, C1-C6 alkyl, and C6-C12 aryl, or taken together with the nitrogen to which they are attached form a cationic center;
L₂ is a second linker that covalently couples the methine carbon to the cationic center;
A₁(=O)—O⁻ is an anionic center, wherein A₁ is selected from the group consisting of C, Si, SO, and POH;
L₃ is a third linker that covalently couples the cationic center to the anionic center;
X⁻ is a counter ion associated with the cationic center;
Y⁺ is a counter ion associated with the anionic center; and
n is an integer from 1 to about 10,000, and
wherein the second polymers have the formula 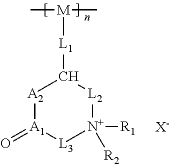
wherein
M is a monomeric repeating unit;
L₁ is a first linker that covalently couples the methine carbon to the repeating unit;
A₂ is O, NR₃, or S, wherein R₃ is selected from the group consisting of hydrogen, C1-C6 alkyl, and C6-C12 aryl;
R₁ and R₂ are independently selected from the group consisting of hydrogen, C1-C6 alkyl, and C6-C12 aryl, or taken together with the nitrogen to which they are attached form a cationic center;
L₂ is a second linker that covalently couples the methine carbon to the cationic center;
L₃ is a third linker that covalently couples the cationic center to A₁;
A₁ is selected from the group consisting of C, Si, SO, and POH;
X⁻ is a counter ion associated with the cationic center;
Y⁺ is a counter ion associated with the anionic center; and
n is an integer from 1 to about 10,000.

11. The surface of claim 8 further comprising a functional molecule covalently coupled to one or more of the polymers of the layer.

12. The surface of claim 8, wherein the substrate is selected from the group consisting of a particle, a drug carrier, non-viral gene delivery system, a biosensor, a membrane, an implantable sensor, a subcutaneous sensor, an implantable device, a contact lens, a heat exchanger, a pipe, a marine structure, a ship hull, a propeller, and a fishing net.

13. A method for converting a surface of a substrate from a zwitterionic polymer surface to a cationic polymer surface, comprising contacting a surface of a substrate having a zwitterionic polymer surface with acidic conditions to provide a cationic polymer surface,
wherein the zwitterionic polymer surface comprises a layer of zwitterionic polymers having the formula 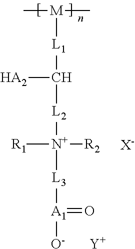
wherein
M is a monomeric repeating unit;
L₁ is a first linker that covalently couples the methine carbon to the repeating unit;
A₂ is O, NR₃, or S, wherein R₃ is selected from the group consisting of hydrogen, C1-C6 alkyl, and C6-C12 aryl;
R₁ and R₂ are independently selected from the group consisting of hydrogen, C1-C6 alkyl, and C6-C12 aryl, or taken together with the nitrogen to which they are attached form a cationic center;
L₂ is a second linker that covalently couples the methine carbon to the cationic center;
A₁(=O)—O⁻ is an anionic center, wherein A₁ is selected from the group consisting of C, Si, SO, and POH;
L₃ is a third linker that covalently couples the cationic center to the anionic center;
X⁻ is a counter ion associated with the cationic center;
Y⁺ is a counter ion associated with the anionic center; and
n is an integer from 1 to about 10,000, and
wherein the cationic polymer surface comprises a layer of cationic polymers having the formula 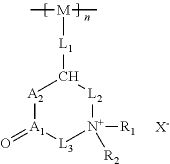
wherein
M is a monomeric repeating unit;
L₁ is a first linker that covalently couples the methine carbon to the repeating unit;
A₂ is O, NR₃, or S, wherein R₃ is selected from the group consisting of hydrogen, C1-C6 alkyl, and C6-C12 aryl;
R₁ and R₂ are independently selected from the group consisting of hydrogen, C1-C6 alkyl, and C6-C12 aryl, or taken together with the nitrogen to which they are attached form a cationic center;
L₂ is a second linker that covalently couples the methine carbon to the cationic center;
L₃ is a third linker that covalently couples the cationic center to A₁;
A₁ is selected from the group consisting of C, Si, SO, and POH;
X⁻ is a counter ion associated with the cationic center;
Y⁺ is a counter ion associated with the anionic center; and
n is an integer from 1 to about 10,000.

14. The method of claim 13 further comprising a functional molecule covalently coupled to one or more of the polymers of the layer.

15. A method for converting a surface of a substrate from a cationic polymer surface to a zwitterionic polymer surface, comprising contacting a surface of a substrate having a cationic surface with aqueous or basic conditions to provide a zwitterionic polymer surface,
wherein the cationic polymer surface comprises a layer of cationic polymers having the formula 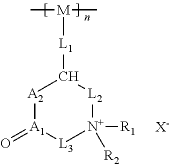
wherein
M is a monomeric repeating unit;
L₁ is a first linker that covalently couples the methine carbon to the repeating unit;
A₂ is O, NR₃, or S, wherein R₃ is selected from the group consisting of hydrogen, C1-C6 alkyl, and C6-C12 aryl;
R₁ and R₂ are independently selected from the group consisting of hydrogen, C1-C6 alkyl, and C6-C12 aryl, or taken together with the nitrogen to which they are attached form a cationic center;
L₂ is a second linker that covalently couples the methine carbon to the cationic center;
L₃ is a third linker that covalently couples the cationic center to A₁;
A₁ is selected from the group consisting of C, Si, SO, and POH;
X⁻ is a counter ion associated with the cationic center;
Y⁺ is a counter ion associated with the anionic center; and
n is an integer from 1 to about 10,000.
wherein the zwitterionic polymer surface comprises a layer of zwitterionic polymers having the formula 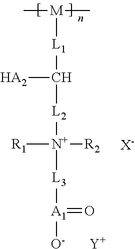
wherein
M is a monomeric repeating unit;
L₁ is a first linker that covalently couples the methine carbon to the repeating unit;
A₂ is O, NR₃, or S, wherein R₃ is selected from the group consisting of hydrogen, C1-C6 alkyl, and C6-C12 aryl;
R₁ and R₂ are independently selected from the group consisting of hydrogen, C1-C6 alkyl, and C6-C12 aryl, or taken together with the nitrogen to which they are attached form a cationic center;
L₂ is a second linker that covalently couples the methine carbon to the cationic center;
A₁(=O)—O⁻ is an anionic center, wherein A₁ is selected from the group consisting of C, Si, SO, and POH;
L₃ is a third linker that covalently couples the cationic center to the anionic center;
X⁻ is a counter ion associated with the cationic center;
Y₊ is a counter ion associated with the anionic center; and
n is an integer from 1 to about 10,000.

16. The method of claim 15 further comprising a functional molecule covalently coupled to one or more of the polymers of the layer.
